(19) United States
(12) Patent Application Publication
(10) Pub. No.: US 2005/0089873 A1
(43) Pub. Date: Apr. 28, 2005

(54) Discrimination of peptides using a molecularly imprinted biosensor

(76) Inventors: Dar-Fu Tai, Hualien Hsien (TW); Chung-Yin Lin, Hualien Hsien (TW); Tzong-Zeng Wu, Hualien Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/690,600

(22) Filed: Oct. 23, 2003

(51) Int. Cl. C12Q 1/68; G06F 19/00; G01N 33/48; G01N 33/50; G01N 33/00

(52) U.S. Cl. 435/6; 436/92

(57) ABSTRACT

Based on the direct formation of molecularly imprinted polymer on gold electrode, the present invention provides a peptide sensor for the detection of low-molecular-weight peptides. A new cross-linking monomer, (N-Acr-L-Cys-NHBn)₂ is employed to attach the surface of the chip and to copolymerize with other monomers. Interestingly, N-benzylacrylamide participating both polymerization and recognition is carried out in an aqueous environment. Using quartz crystal microbalance detection, short peptides can be monitored by their interaction with plastic antibodies specific for the target peptides. The selectivity of molecularly imprinted polymer and the sensitivity of such artificial biosensors have collaborated to differentiate traces of oxytocin and vasopressin to the ng/ml scale.

### BACKGROUND OF THE INVENTION

Molecular imprinting (MIPs) is a process for synthesizing organic polymers that contain recognition sites for small molecules. The imprinting process consists of a template molecule that organizes functional and cross-linking polymerizable monomers during the polymerization process. The template is extracted from the insoluble network material leaving behind domains that are complementary in size, shape, and functional group orientation to the template molecule. The preparation of molecularly imprinted polymers as the stationary phase for selective separation of amino acids and small peptides has been known. Some of these systems have utilized protected peptides in organic solvents. These formulations employ free radical polymerization and rely on the use of hydrogen-bonding interactions between the template and functional monomers as the selectivity-providing interaction.

Direct detection of peptide-macromolecule interaction is rare and is currently under investigation. The preparation of artificial binding sites for such peptides may provide insight into recognition processes. Examples include sensing of enkephalins, tripeptides, helical peptides, oxytocin and its derivatives, by imprinted macromolecular receptor. A recent progress was the recognition of His-peptides using peptide-metal interactions. These artificial receptors may also facilitate the screening of peptide mixtures, proteins or assist in the evaluation of peptidomimetics that can be used to either enhance or inhibit receptor responses.

The present invention for creating peptide receptors using molecular imprinting takes advantage of quartz crystal microbalance (QCM). The QCM is a kind of bulk-acoustic wave (BAW) resonator, as derived by Saurbrey. In 1980, Konash and Bastiaans developed an apparatus—QCM—fixed between two spacers allowing the liquid to flow through one side with the other side in contact with air. This permits the oscillation to occur in the liquid and measuring the QCM in liquid. Due to the high sensitivity, simple operation, easy interpretation and “real-time” measurement, QCM allows the label-free detection of molecules with applications to the study of kinetics, peptide binding to immobilized oligonucleotides, protein binding to immobilized receptors, medical diagnosis the detection of pathogenic microorganisms, and other molecular discrimination events. MIP-QCM sensor has been reported for the detection of (S)-propanolol and terpenes in organic solvent.

### SUMMARY OF THE INVENTION

The objective of the present invention is to provide a method for discriminating a peptide.

In the present invention, the method for discriminating a peptide includes steps of: (a) providing an organic compound which serves as an adsorbent, a cross-linker and a monomer; (b) adsorbing said organic compound on a chip to form a single layer; and (c) associating monomers with double bonds and template molecules to said chip to form a molecularly imprinted membrane thereon by polymerization.

The aforementioned organic compound is usually a derivative of cystine, and preferably includes L-cystine, D-cystine, racemic cystine, L-homocystine, D-homocystine or racemic homocystine, for example, (Acr-Cys-NHBn)₂, (Acr-Cys-NHΦ)₂, (Macr-Cys-NHBn)₂, (Macr-Cys-NHΦ)₂, (Acr-hCys-NHBn)₂, (Acr-hCys-NHΦ)₂, (Macr-hCys-NHBn)₂ and (Macr-hCys-NHΦ)₂; wherein hCys is homocystine, Φ is phenyl, Macr is methacryl, and (Acr-Cys-NHBn)₂ is preferred. Alternatively, (Macr-AA-NHBn)₂, (Macr-AA-NHΦ)₂, methacrylamide, methacrylic acid, N-benzyl-methacrylamide, (Acr-AA-NHBn)₂, (Acr-AA-NHΦ)₂, acrylamide, acrylic acid or N-benzyl-acrylamide, wherein AA is L, D, or racemic amino acid, Φ is phenyl and Macr is methacryl, also can be used in the present invention.

The template molecule can be amino acid, nucleo acid, carbohydrate, lipid or peptide such as oxytocin and vasopressin.

In step (b), the organic compound can be adsorbed on said chip by dissolving (Acr-Cys-NHBn)₂ in a mixture of acetonitrile (10 ml) and DMF (0.1 ml), which is then deposited on said chip therein.

The monomers with double bonds in step (c) primarily include acrylic acid, acrylamide and N-benzylacrylamide, which are preferably added at a molar ratio 1:1:2.

In step (c), polymerization is preferably carried out by either irradiating with light at 350 nm for 6 hours or heating at 50˜100° C. to completion.

In general, the method for discriminating a peptide in accordance with the present invention, is preferably carried out by adsorbing (Acr-Cys-NHBn)₂ on a chip to form a single layer; and then associating acrylamide, acrylic acid and N-benzyl-acrylamide to the chip to form a molecularly imprinted membrane through radical polymerization.

Other objects, advantages, and novel features of the invention will become more apparent from the following detailed description when taken in conjunction with the accompanying drawings.

### BRIEF DESCRIPTION OF THE FIGURES

The present invention will be better understood by referring to the accompanying drawings, wherein:

FIG. 1 shows synthesis of (N-Acr-L-Cys-NHBn)₂;

FIG. 2 is schematic representation of the peptide imprinting process;

FIG. 3 shows the frequency changes of oxytocin and vasopressin obtained using oxytocin-imprinted QCM;

FIG. 4 shows the frequency changes of oxytocin and vasopressin obtained using vasopressin-imprinted QCM;

FIG. 5 shows the binding effects of oxytocin-imprinted QCM; and

FIG. 6 shows the binding effects of vasopressin-imprinted QCM.

### DETAILED DESCRIPTION OF THE PREFERRED EMBODIMENTS

The present invention provides protocols for molecular imprinting that create macromolecular receptors for small peptides. Oxytocin is a nonapeptide that is synthesized in hypothalamic neurons and transported down axons of the posterior pituitary for secretion into blood. In the preferred embodiments of the present invention, oxytocin and another nonapeptide vasopressin with amino acid sequence shown in Table 1 were chosen as the template target for capturing molecular imprint sites. The availability of the water soluble form of both peptides, which could be used to establish the specificity of the interaction.

TABLE 1

| | Peptides | Amino Acid Sequence |
|---|---|---|
| Examples 1 and 2 | Oxytocin | 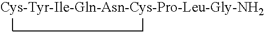 |
| Example 3 | Vasopressin | 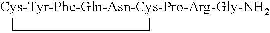 |
| Comparative | Angiotension | Asp-Arg-Val-Tyr-Ile-His-Pro-Phe |
| Example 1 | II | |
| Comparative | Bradykinin | Arg-Pro-Pro-Gly-Phe-Ser-Pro-Phe-Arg |
| Example 2 | | |
| Comparative | 15-mer | Thr-Glu-Leu-Arg-Tyr-Ser-Trp-Lys-Thr- |
| Example 3 | peptide | Trp-Gly-Lys-Ala-Lys-Met |

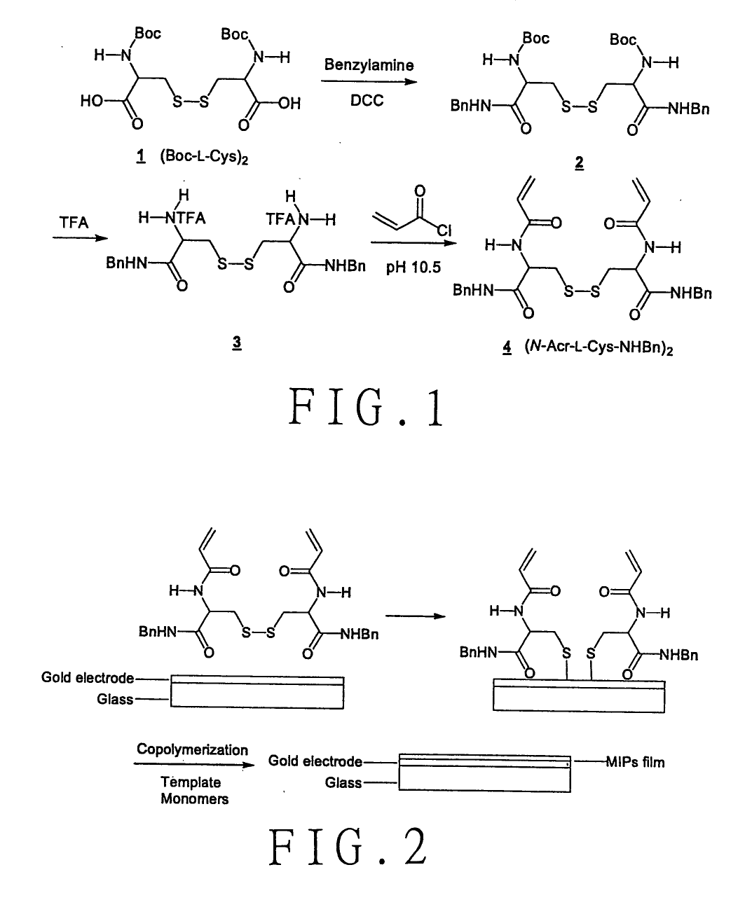

FIG. 1 shows synthesis of (N-Acr-L-Cys-NHBn)₂. A new cross-linking monomer in neutral form, containing chiral center as well as disulfide bond was designed and prepared. As shown in FIG. 1, synthesis of (N-Acr-L-Cys-NHBn)₂ is straightforward with a total yield of 50% from N,N′-diBoc-L-cystine ((Boc-L-Cys)₂).

FIG. 2 is schematic representation of the peptide imprinting process in accordance with the present invention. FIG. 2 also illustrates the present invention for preparing highly cross-linked polyacrylamides containing binding sites, which incorporate a (N-Acr-L-Cys-NHBn)₂-Au complex. The QCM employed in this work consisted of a disk of crystalline quartz with gold electrodes on the upper and lower surfaces. The use of water in the polymer synthesis and recognition steps has obvious advantages over organic systems. Although protic solvents such as alcohols and water is compatible with free radical polymerization, they have been largely excluded from use in imprinting due to their abilities to compete with hydrogen-bonding interactions. However, as lack of solubility of peptides in organic media and more subtle effects such as peptide conformation, a water/acetonitrile mixture was made the solvent of choice.

The polymerizable (N-Acr-L-Cys-NHBn)₂-Au complex was prepared by combining aqueous solutions of (N-Acr-L-Cys-NHBn)₂ on a 4.5 mm diameter gold electrodes. The disulfide functional group was used as a “glue” to attach (N-Acr-L-Cys-NHBn)₂ to the electrode, an asymmetric molecule to provide chirality to the QCM surface and a cross-linker to copolymerize with other monomers. The benzylamide of (N-Acr-L-Cys-NHBn)₂ also prevented displacement of the polymer by self-assembly of N-benzylacrylamide (BAA) or template to form a hydrophobic layer. All the monomers and cross-linker were thus attached to the surface to formulate MIPs in a more organized manner after copolymerization.

To avoid imbedding too much amounts of the template, copolymerization of the (N-Acr-L-Cys-NHBn)₂-Au complex was carried out without adding other cross-linking monomer. The polymerization complex was then formed by irradiation with BAA, acrylic acid, acrylamide and template in a water/acetonitrile mixture. The polymer, which was formed as a thin film, was washed with phosphate buffer to remove template, followed by a wash with acetonitrile and drying.

The specificities of the above MIP-grafted QCMs were evaluated by injecting oxytocin or vasopressin solutions at different concentrations, respectively.

More detailed procedures for producing the biosensor of the present invention are described in the following examples. In these examples, (Boc-L-CYS)₂, acrylic acid, acrylamide, oxytocin, angiotensin II, bradykinin and vasopressin, were obtained from Sigma-Aldrich (St. Louis, Mo.). N-benzylacrylamide was purchased from Lancaster (Lancashire, UK). The buffer used for all experiments was PBS (20 mM NaH₂PO₄, pH 7.0). The QCM was obtained from Tai-Tien Electronic Co. (Taipei, Taiwan) with a reproducibility of ±1 Hz. The QCM consisted of an 8 mm diameter disk made from an AT cut 9 MHz quartz crystal with a gold electrodes on both sides (diameter: 4.5 mm, area: 15.9 mm²) of the crystal.

### EXAMPLE 1

(1) Synthesis of (N-Acr-L-Cys-NHBn)₂

Synthesis of (N-Acr-L-Cys-NHBn)₂ is straightforward with a total yield of 50% from N,N′-diBoc-L-cystine ((Boc-L-CYS)₂).

(2) Preparation of Imprinted Polymer-Coated QCM

The QCM disks were immersed in a 10 μM solution of (N-Acr-L-Cys-NHBn)₂ in HPLC-grade acetonitrile for 16 hrs, then rinsed exhaustively with acetonitrile and then dried under vacuum. A solution of acrylic acid (55 μmol), acrylamide (55 μmol), N-benzylacrylamide (110 μmol), and 3 μmol of template oxytocin were mixed in 0.3 ml of solution (acetonitrile/water=1:1). The above BAA, acrylic acid and acrylamide are at a mole ratio of 2:1:1. After depositing 4 μl of the aliquot on top of the (N-Acr-L-Cys-NHBn)-gold electrode, the chip was placed horizontally into a 20 ml vial containing acetonitrile (3 ml). The vial was screwed tightly and irradiated with UV-light at 350 nm for 6 hrs. The polymer, which was formed as a thin film on the gold surface, was washed with 20 mM phosphate buffer (pH=3-4) to remove 70 to 80% of the template. This was followed by a wash with acetonitrile and drying. The thickness of the polymer films were measured as 92±15 nm by using a surface profiler from Veeco Inc. (Dekatak³ ST). The frequency shifted −750±44 Hz after coating with (N-Acr-L-Cys-NHBn)₂ and shifted further lower to −3400±800 Hz after copolymerization. It shifted back 300±50 Hz after the removal of the template.

(3) Biosensor System

The flow injection system containing a HPLC pump (Model L7110, Hitachi, flow rate=0.1 ml min⁻¹), home-build flow cell, sample injection valve (Model 1106, OMNIFIT), home-built oscillation circuit (including oscillator and frequency counter) and a personal computer. The polymer coated QCM was fixed between two O-ring and inserted into the flow-cell. Only one side of the QCM was in contact with the liquid. PBS was used for circulating, washing and testing.

### EXAMPLE 2

Repeat procedures of EXAMPLE 1, but BAA, acrylic acid and acrylamide are at a mole ratio of 1:1:1 for preparation of imprinted polymer-coated QCM.

### EXAMPLE 3

Repeat procedures of EXAMPLE 1, but oxytocin is replaced with vasopressin for preparation of imprinted polymer-coated QCM.

### COMPARATIVE EXAMPLE 1

Repeat procedures of EXAMPLE 1, but oxytocin is replaced with Angiotension II for preparation of imprinted polymer-coated QCM.

### COMPARATIVE EXAMPLE 2

Repeat procedures of EXAMPLE 1, but oxytocin is replaced with Bradykinin for preparation of imprinted polymer-coated QCM.

### COMPARATIVE EXAMPLE 3

Repeat procedures of EXAMPLE 1, but oxytocin is replaced with 15-mer peptide for preparation of imprinted polymer-coated QCM.

Evaluation of MIP-Grafted QCMs

Binding tests were performed to evaluate uptake of the template and non-template peptides. Aqueous solutions (PBS, pH=7) were flowed through the system. After equilibration, 100 μl of aqueous solutions of the tested peptide were injected and the change of frequency was measured by QCM. Binding isotherms were obtained for the template peptide (oxytocin) as well as vasopressin.

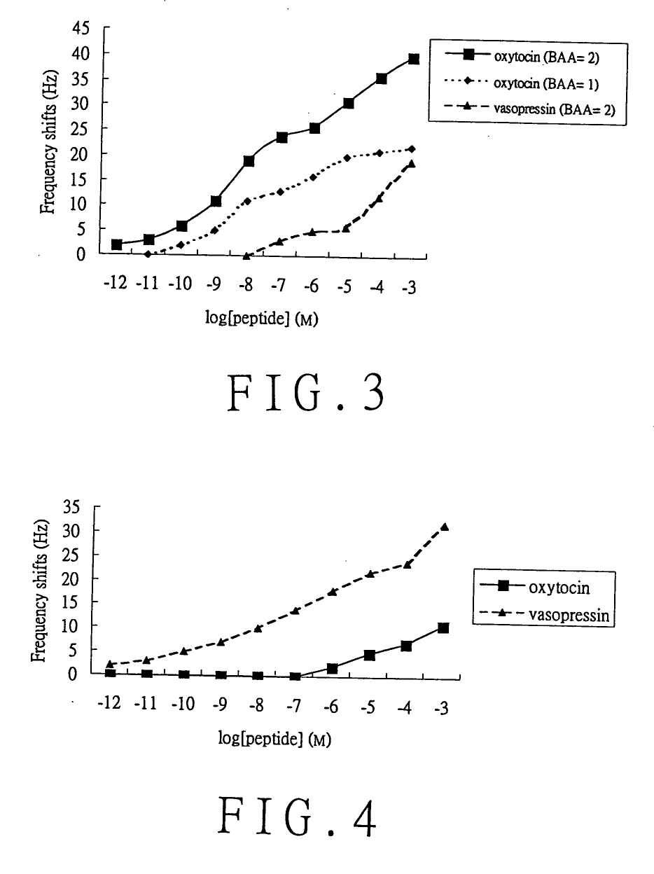

FIGS. 3 and 4 show the frequency changes of oxytocin and vasopressin obtained using oxytocin-imprinted QCM and vasopressin-imprinted QCM, respectively. As shown in FIGS. 3 and 4, the adsorption of non-template peptides was not observed until the concentration of other peptides reached 1 ng/ml. The frequency shifts of three other peptides, angiotensin II, bradykinin, and 15-mer peptide were compared in the same concentration. No trace was detected at 1 ng/ml. However, nonspecific adsorption of these peptides began to be visible when the concentration reached the level of 1 μg/ml.

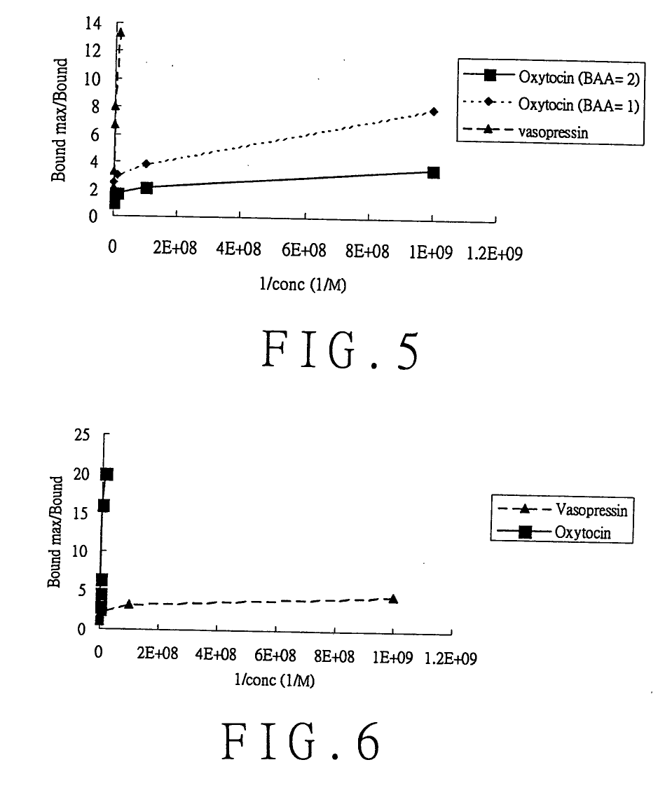

To clearly demonstrate the binding abilities of MIPs, Bmax is set as the maximum frequency shift observed and B is the frequency shift obtained at the indicated concentration of peptide. FIGS. 5 and 6 show the binding effects of oxytocin-imprinted QCM and vasopressin-imprinted QCM, respectively. Thus, K_{d} were calculated from the slope of curves. The best oxytocin MIP's K_{d} value for oxytocin was about 1.1*10⁻⁸ M (FIG. 5). The best vasopressin MIP's K_{d} value for vasopressin was about 2.0*10⁻⁸ M (FIG. 6). In general, MIP demonstrated a marked 10˜100 times enhancement in K_{d} value toward template-peptide higher than their nonspecific adsorptions to nontemplate-peptide.

The peptide recognition sites were formed by incorporating two types of interactions that are established during the polymerization. One consists of ionic binding between acrylic acid and N-terminal of the peptide. This binding is compromised by water or other protic solvents. The second bonding frame comprises multiple weaker interactions between the network polymer chains and the imprinting peptide molecule. FIG. 3 shows that the hydrophobic interactions between the peptide and N-benzylacrylamide are very important. Without N-benzylacrylamide, the polymer matrixes that are developed during the polymerization are not sufficient to provide sequence selectivity between the imprinted peptide and other amino acid sequences. Compared to only one fold of N-benzylacrylamide, the frequency shifts were larger as the monomer ratio is 2:1:1.

In conclusion, the present invention shows that it is possible to directly and sensitively discriminate peptides, using a combination technology of molecular imprinting and QCM. Interestingly, N-benzylacrylamide participates both polymerization and recognition is carried out in an aqueous environment. Therefore, the present invention provided protocols for creating macromolecular receptors for peptides using molecular imprinting. This system may be helpful in understanding the modes of peptide recognition processes. They may also find use as artificial sensors for screening of peptides and peptidomimetics.

1. A method for discriminating a peptide, comprising steps of:
(a) providing an organic compound which serves as an adsorbent, a cross-linker and a monomer;
(b) adsorbing said organic compound on a chip to form a single layer; and
(c) associating monomers with double bonds and template molecules to said chip to form a molecularly imprinted membrane thereon by polymerization.
(d) detecting by a quartz crystal microbalance (QCM) or a surface plasma resonance (SPR) equipped with a flow injection system.

2. The method of claim 1, wherein said organic compound is a derivative of cystine or homocystine.

3. The method of claim 2, wherein said derivative of cystine or homocystine comprises L-cystine, D-cystine, racemic cystine, L-homocystine, D-homocystine or racemic homocystine.

4. The method of claim 2, wherein said derivative of cystine is (Acr-Cys-NHBn)₂, (Acr-Cys-NHΦ)₂, (Macr-Cys-NHBn)₂, (Macr-Cys-NHΦ)₂, (Acr-hCys-NHBn)₂, (Acr-hCys-NHΦ)₂, (Macr-hCys-NHBn)₂ or (Macr-hCys-NHΦ)₂; wherein hCys is homocystine, Φ is phenyl, and Macr is methacryl.

5. The method of claim 1, wherein said monomers are (Macr-Cys-NHBn)₂, (Macr-AA-NHBn)₂, (Macr-Cys-NHΦ)₂, (Macr-AA-NHΦ)₂, (Acr-hCys-NHBn)₂, (Acr-hCys-NHΦ)₂, (Macr-hCys-NHBn)₂, (Macr-hCys-NHΦ)₂, methacrylamide, methacryic acid, N-benzyl-_methacrylamide, (Acr-Cys-NHBn)₂, (Acr-AA-NHBn)₂, (Acr-Cys-NHΦ)₂, (Acr-AA-NHΦ)₂, acrylamide, acrylic acid or N-benzyl-acrylamide; wherein AA is L-, D- or racemic amino acid, Φ is phenyl and Macr is methacryl.

6. The method of claim 1, wherein said template molecule is amino acid, nucleo acid, carbohydrate, lipid or peptide.

7. The method of claim 6, wherein said peptide is oxytocin.

8. The method of claim 6, wherein said peptide is vasopressin.

9. The method of claim 1, wherein said organic compound is adsorbed on said chip by dissolving (Acr-Cys-NHBn)₂ in a mixture of acetonitrile (10 ml) and DMF (0.1 ml), which is then deposited on said chip therein.

10. The method of claim 1, wherein said monomers with double bonds are acrylic acid, acrylamide and N-benzylacrylamide which are added at a molar ratio 1:1:2.

11. The method of claim 1, wherein said polymerization is carried out by either irradiating with light at 350 nm for 6 hours or heating at 50˜100° C. to completion.

12. A method for discriminating a peptide, using a combination technology of molecular imprinting and QCM, in which (Acr-Cys-NHBn)₂ is adsorbed on a chip to form a single layer; and then acrylamide, acrylic acid, N-benzyl-acrylamide are associated to said chip to form a molecularly imprinted membrane by radical polymerization.
